SEQUENCE LISTING (21 sequences)

SEQ ID NO: 1 - DNA, 16 bp, Mycobacterium tuberculosis
gaccgtgcggatcttg

SEQ ID NO: 2 - DNA, 18 bp, Mycobacterium tuberculosis
gctgcacatggagatcac

SEQ ID NO: 3 - DNA, 24 bp, Mycobacterium tuberculosis
cggatttcggtgccgtcgtctttg

SEQ ID NO: 4 - DNA, 19 bp, Mycobacterium tuberculosis
gtgcagaaggtctgtaacg

SEQ ID NO: 5 - DNA, 18 bp, Mycobacterium tuberculosis
ggacacagccttgttcac

SEQ ID NO: 6 - DNA, 17 bp, Mycobacterium tuberculosis
caccgacgcgaaagtcg

SEQ ID NO: 7 - DNA, 23 bp, Lactococcus lactis
ccttaggtattcgtatggttgac

SEQ ID NO: 8 - DNA, 18 bp, Lactococcus lactis
acccgcttgaacagagta

SEQ ID NO: 9 - DNA, 25 bp, Lactococcus lactis
caaccactccaccagttacgctgct

SEQ ID NO: 10 - DNA, 1962 bp, Mycobacterium tuberculosis
tcacttcttgcctttgtccccggcggcatcggtggacaatgccgcgacgaaagcctcctgtggcacctcgacgcgcccgatggtcttcatccgcttcttgccttccttctgcttctccagcagcttgcgtttgcgcgtgatgtcgccgccgtagcacttggacaacacgtccttgcggatcgcgcggatgttttcgcgggcaatgattttcgatccgatggcggcctgcaccggcacctcgaactgctggcgcgggatcagctccttgagtttggtggtcatcttgttgccgtaggcatacgccgtgtccttgtgcacgatcgcgctgaacgcatccaccgcctcgccctgcagcaggatgtcgaccttgaccagcgcggcctcctgttcgccggcctcctcgtagtcgaggctggcatagccgcgggtgcgcgatttcagtgcgtcgaagaagtcgaagatgatctcgccgagcggcatggtgtagcgcagttccacccgctcgggggagagatagtccatgccgcccaactcgccgcggcgcgactggcacagctccatgatggtgccgatgaactcgctgggcgcgatgatggtggtcttgacgacgggctcgtagaccgtgcggatcttgccctccggccagtccgacggattggtcacccggatttcggtgccgtcgtctttgtgcacccgatacaccacattgggtgaggtcgagatcaggtccaggccgaactcgcgctcaaggcgctcacgggtgatctccatgtgcagcaggcccaagaaaccgcaccggaacccaaaacccagcgccaccgaggtttccggctcataggtcaaggccgcgtcgttgagctgcagcttgtccagggcgtcgcgcaggttcgggtagtccgaaccgtcgaccggatacaaccccgagtagaccatcggtttgggctcacggtagccggtcaacgcttcggcggcagccccgcgggcccgggagaggctggtcacggtgtcgcccaccttggactggcggacgtccttgacgccggtgatcaggtaacccacctcgccgacaccgaggccctcacacggtttcggctcgggtgagacgatgccgacctcaagcagctcgtgggtggcgccggtggacatcatcatgatgcgctcacgggggctgatcttgccgtcgacgacgcggacgtaggtcaccactccgcggtagatgtcgtaaacggagtcgaaaatcattgcgcgggtaggtgcctcggcgtcgccctgagggggcggcacctgtcggaccacctcgtcgagcaggtcggacacgccttcgccggttttgccggacacccgcaacacctcggccggctcgcagccgatgatgtgtgccatctcggcggcgtaacggtccgggtcggccgcgggcaggtcgatcttgttgagcaccgggatgatgtgcaggtcgcggtccaacgccaggtagaggttcgccagcgtctgcgcctcgatgccttgcgcggcatcgaccaacagcaccgcaccctcgcaagcctccagcgcacgcgagacttcgtaggtgaagtcgacatggcccggggtgtcgatcagatgcagcacgtagtcggtcttgtcgacccgccagggtagccgcacattctgggccttgatggtgatgccgcgttcccgctcgatgtccatccgatccaagtactgggcccgcatagagcgttcgtcgaccacgccggtgagctgcagcatccggtcggccaacgttgacttgccgtggtcgatgtgggcgatgatgcaaaagttcctaatctgcgccggcgcagtgaaggttttgtcggcgaaactgctgatgggaatctcctggagcgggggttgacgggtatccagggtatccgcgtcgggcagctgcgacccaatcgcgctcggtcgatcgcgtctatgctgcgagcatggcgtccgcac

SEQ ID NO: 11 - DNA, 2145 bp, Mycobacterium tuberculosis
atgggtaaaaacgaggccagaagatcggccctggcgcccgatcacggtacagtggtgtgcgaccccctgcggcgactcaaccgcatgcacgcaacccctgaggagagtattcggatcgtggctgcccagaaaaagaaggcccaagacgaatacggcgctgcgtctatcaccattctcgaagggctggaggccgtccgcaaacgtcccggcatgtacattggctcgaccggtgagcgcggtttacaccatctcatttgggaggtggtcgacaacgcggtcgacgaggcgatggccggttatgcaaccacagtgaacgtagtgctgcttgaggatggcggtgtcgaggtcgccgacgacggccgcggcattccggtcgccacccacgcctccggcataccgaccgtcgacgtggtgatgacacaactacatgccggcggcaagttcgactcggacgcgtatgcgatatctggtggtctgcacggcgtcggcgtgtcggtggttaacgcgctatccacccggctcgaagtcgagatcaagcgcgacgggtacgagtggtctcaggtttatgagaagtcggaacccctgggcctcaagcaaggggcgccgaccaagaagacggggtcaacggtgcggttctgggccgaccccgctgttttcgaaaccacggaatacgacttcgaaaccgtcgcccgccggctgcaagagatggcgttcctcaacaaggggctgaccatcaacctgaccgacgagagggtgacccaagacgaggtcgtcgacgaagtggtcagcgacgtcgccgaggcgccgaagtcggcaagtgaacgcgcagccgaatccactgcaccgcacaaagttaagagccgcacctttcactatccgggtggcctggtggacttcgtgaaacacatcaaccgcaccaagaacgcgattcatagcagcatcgtggacttttccggcaagggcaccgggcacgaggtggagatcgcgatgcaatggaacgccgggtattcggagtcggtgcacaccttcgccaacaccatcaacacccacgagggcggcacccacgaagagggcttccgcagcgcgctgacgtcggtggtgaacaagtacgccaaggaccgcaagctactgaaggacaaggaccccaacctcaccggtgacgatatccgggaaggcctggccgctgtgatctcggtgaaggtcagcgaaccgcagttcgagggccagaccaagaccaagttgggcaacaccgaggtcaaatcgtttgtgcagaaggtctgtaacgaacagctgacccactggtttgaagccaaccccaccgacgcgaaagtcgttgtgaacaaggctgtgtcctcggcgcaagcccgtatcgcggcacgtaaggcacgagagttggtgcggcgtaagagcgccaccgacatcggtggattgcccggcaagctggccgattgccgttccacggatccgcgcaagtccgaactgtatgtcgtagaaggtgactcggccggcggttctgcaaaaagcggtcgcgattcgatgttccaggcgatacttccgctgcgcggcaagatcatcaatgtggagaaagcgcgcatcgaccgggtgctaaagaacaccgaagttcaggcgatcatcacggcgctgggcaccgggatccacgacgagttcgatatcggcaagctgcgctaccacaagatcgtgctgatggccgacgccgatgttgacggccaacatatttccacgctgttgttgacgttgttgttccggttcatgcggccgctcatcgagaacgggcatgtgtttttggcacaaccgccgctgtacaaactcaagtggcagcgcagtgacccggaattcgcatactccgaccgcgagcgcgacggtctgctggaggcggggctgaaggccgggaagaagatcaacaaggaagacggcattcagcggtacaagggtctaggtgaaatggacgctaaggagttgtgggagaccaccatggatccctcggttcgtgtgttgcgtcaagtgacgctggacgacgccgccgccgccgacgagttgttctccatcctgatgggcgaggacgtcgacgcgcggcgcagctttatcacccgcaacgccaaggatgttcggttcctggatgtctaa

SEQ ID NO: 12 - DNA, 20 bp, Mycobacterium tuberculosis
cgtagaccgtgcggatcttg

SEQ ID NO: 13 - DNA, 21 bp, Mycobacterium tuberculosis
cctgctgcacatggagatcac

SEQ ID NO: 14 - DNA, 22 bp, Mycobacterium tuberculosis
gcagaaggtctgtaacgaacag

SEQ ID NO: 15 - DNA, 21 bp, Mycobacterium tuberculosis
cgaggacacagccttgttcac

SEQ ID NO: 16 - DNA, 23 bp, Artificial sequence
  Plasmid based on pUC57 serving as extraction control
aattcctcagggattattgtggc

SEQ ID NO: 17 - DNA, 24 bp, Artificial sequence
  Plasmid based on pUC57 serving as extraction control
cacaaccagtatgcaacatcaacc

SEQ ID NO: 18 - DNA, 25 bp, Artificial sequence
  Probe to detect amplif product in Plasmid based on pUC57 as extraction control
aacctcttgaactgacagcgtgtgc

SEQ ID NO: 19 - DNA, 527 bp, Artificial sequence
  Plasmid comprising lepA sequence of MTC
gcggccgcctggggttggggtggagtctctgacggcttcttccgtcttgacgcactaaacccttcagctcttggtactggtggcggcgcaggcggcttcaacggttaccaaagtgctgttgtaggcatcaaaccttagtatggaaatgcatggtggtcttgacgacgggctcgtagaccgtgcggatcttgccctccggccagtccgacggattggtcacccggatttcggtgccgtcgtctttgtgcacccgatacaccacattgggtgaggtcgagatcaggtccaggccgaactcgcgctcaaggcgctcacgggtgatctccatgtgcagcaggcccaagaaaccgcaccggaacccgccagcagggctcttcgtcagctcccactcgtagccgtacaggatctcgaggaaactgttgtcccatttcgtcggggtgttcgtccatacgacctcgatgccggtggtgatcgcgtccttaccggttccggtgccatacgagctcttccagcccaagcccatctgc

SEQ ID NO: 20 - DNA, 403 bp, Artificial sequence
  Plasmid comprising gyrB sequence
gcggccgcgggcaacaccgaggtcaaatcgtttgtgcagaaggtctgtaacgaacagctgacccactggtttgaagccaaccccaccgacgcgaaagtcgttgtgaacaaggctgtgtcctcggcgcaagcccgtatcgcggcacgtaaggcacgagagttggtgcggcgtaagagcgccaccgacatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgc

SEQ ID NO: 21 - DNA, 443 bp, Artificial sequence
  Plasmid based on pUC57 serving as extraction control
gctaatacgactcactatagggagatagcatttaggtgacactatagaagttagtacctgggaagcaagccgcggaaatgatcagaagacgtgcgaattcctcagggattattgtggccacgaaggataacgttaaaaccgttgattctttcatgatgaattttgggaaaagcacacgctgtcagttcaagaggttattcattgatgaagggttgatgttgcatactggttgtgttaattttcttgtggcgatgtcattgtggtaagatttcacgtcctgccttaggtattcgtatggttgacctatctcaattatcaacaaatgatagttctcaactgaaattacctagcagcgtaactggtggagtggttgtctactctgttcaagcgggtcttcctgctgccacagctggaaaaaaaaaaaaaaaaaaaaaaaaaaaaaa

(19) United States
(12) Patent Application Publication
(10) Pub. No.: US 2015/0057172 A1
(43) Pub. Date: Feb. 26, 2015

(54) REAL-TIME PCR DETECTION OF MYCOBACTERIUM TUBERCULOSIS COMPLEX

(71) Applicant: VELA OPERATIONS PTE. LTD., Singapore (SG)

(72) Inventor: Elian Rakhmanaliev, Singapore (SG)

(21) Appl. No.: 14/382,747

(22) PCT Filed: Mar. 6, 2013
(86) PCT No.: PCT/IB2013/051769

§ 371 Date: Sep. 3, 2014

(30) Foreign Application Priority Data
Mar. 6, 2012 (GB) 1203903.8

(51) Int. Cl. C12Q 1/68

(52) U.S. Cl. CPC C12Q 1/689; C12Q 2600/16; C12Q 2600/136
USPC 506/9; 506/16

(57) ABSTRACT

The present invention relates to assays, diagnostic kits and methods for the simultaneous real-time PCR detection of bacteria belonging to the MTR and/or Mycobacterium tuberculosis and methods, compositions, kits and assays allowing differentiation between MTC species other than Mycobacterium tuberculosis.

### FIELD OF THE INVENTION

The present invention relates to the detection and diagnosis of species belonging to the Mycobacterium tuberculosis Complex (MTC) (M. tuberculosis, M. africanum, M. bovis, M. bovis BCG, M. canetti and M. microti) and the differentiation between M. tuberculosis and other MTC species.

### BACKGROUND

Rapid identification of pathogens causing infectious diseases is an important goal in diagnostic technology. The challenge facing the clinician is to identify the pathogenic species and to affirm the potential efficacy of standard microbial treatments as early as possible after infection. Wrong laboratory results and time-consuming methods to identify the causative pathogen are frequently associated with an exacerbation of the disease or wrong treatments. This not only results in increased costs to the health system, but also in potentially life-threatening situations and other disadvantages for the patient.

Mycobacterial species are responsible for a significant number of tuberculosis-associated morbidity and mortality in humans, but Mycobacterium tuberculosis (Mtb) is responsible for millions of infections and fatalities throughout the world. The responsible bacterium is a gram-positive, non-motile, pleomorphic rod related to Actinomyces.

Because tuberculosis is highly contagious, rapid diagnosis of the disease is of utmost importance. Classical methods for the identification of mycobacteria rely on staining specimen for acid-fast bacilli followed by culturing and biochemical testing. These difficult techniques render the identification and quantification of MTC complex pathogen very inefficient. There exists a need in the art for rapid and cost-efficient tests for the diagnosis of tuberculosis, in particular for the detection of MTC complex species and the differentiation between M. tuberculosis and other MTC species, as M. tuberculosis is by far the most virulent human mycobacterial pathogen.

The present invention addresses this requirement and provides assays, kits and methods for the detection and diagnosis of infection and differentiation between M. tuberculosis and other MTC species with very high specificity and sensitivity, e.g. wherein only about 10 to 100 copies of target nucleic acids can be detected, whereas nucleic acids from other microorganisms should not be detected.

The present invention takes advantage of nucleotide polymorphisms at position 665, 666, 667, 668, 670, 675, 676, 685 and 687 of the coding region of the lepA gene to design a probe which is specific to MTC species only and to distinguish MTC from other species. Furthermore, the use of a nucleotide polymorphism at position 877 of the coding region of the gyrB gene is used to design probes specific for M. tuberculosis and to distinguish the same from other MTC species.

### SUMMARY OF THE INVENTION

Accordingly, the present invention relates to a set of nucleic acids, useful for detection of MTC and distinction between M. tuberculosis and other MTC species.

In a first embodiment, the first set of primers comprises sequences shown in SEQ ID Nos: 1 and 2 or in SEQ ID Nos: 12 and 13, the second set of primers comprises SEQ ID Nos: 4 and 5 or in SEQ ID Nos: 14 and 15, preferably the first probe has a sequence of SEQ ID No: 3 and the second probe has a sequence of SEQ ID NO: 6. Also covered by the present invention are complementary sequences or sequences having at least 80%, 85%, 90% or 95% homology or identity with any of the sequences in SEQ ID Nos.: 1 to 6 or in SEQ ID Nos.: 12 to 15.

The present invention also relates to a method for the detection of nucleic acids derived from bacteria belonging to the MTC and to the specific distinction of Mycobacterium tuberculosis from other bacteria of the MTC in a biological sample from a patient, comprising:
a) providing a biological sample from a patient;
b) extracting nucleic acids (DNA and/or RNA) from the biological sample;
c) carrying out a PCR or RT-PCR using oligonucleotides according to the present invention; and
d) detecting amplification products specific for MTC and/or Mycobacterium tuberculosis, wherein the presence of respective amplification products is indicative of the presence of MTC bacteria and/or Mycobacterium tuberculosis in the biological sample.

In an alternative method, instead of carrying a RT-PCR, the method comprises a step of reverse-transcription and a step of PCR amplification.

The present invention further concerns the use of a set of nucleic acids according to the present invention for detecting bacteria belonging to MTC and/or Mycobacterium tuberculosis.

It further concerns a method of detecting or diagnosing MTC and/or Mycobacterium tuberculosis using a set of nucleic acids according to the present invention.

In addition, it concerns a set of nucleic acids according to the present invention for preparing a diagnostic kit useful for detecting MTC and/or Mycobacterium tuberculosis. Optionally, the kit further comprises other components such as controls for the extraction (i.e. control nucleic acids, primers and probes), DNA polymerase, reverse-transcriptase, RNase inhibitors, dNTPs and a PCR and/or RT-buffers.

### SHORT SUMMARY OF PREFERRED EMBODIMENTS

Some of the preferred embodiments of the invention are depicted below:
i. A method for the detection of the presence or absence of at least one nucleic acid of bacteria belonging to the MTC and/or Mycobacterium tuberculosis in a biological sample, wherein the method comprises conducting real-time PCR.
ii. The method according to (i) further comprising isolating (extracting) nucleic acids from the biological sample and performing a reverse transcription step.
iii. The method according to any one of (i) or (ii), wherein primer sets that are specific for MTC and/or Mycobacterium tuberculosis are used.
iv. The method according to any one of (i) to (iii), wherein the Mycobacterium MTC and/or Mycobacterium tuberculosis-specific primer set comprises oligonucleotide sequences set forth in SEQ ID Nos: 1 and 2 or in SEQ ID Nos: 12 and 13, or subcombinations thereof (e.g. SEQ ID Nos: 1 and 13), and SEQ ID Nos: 4 to 6 or in SEQ ID Nos: 14 and 15 or subcombinations thereof (e.g. SEQ ID Nos.: 4 and 15) or complements of any of the sequences, or sequences having at least 80%, preferably 90%, more preferably at least 95%, 96%, 97%, 98%, 99% homology or complements thereof.
v. The method according to any one of (i) to (iv), wherein at least one probe specifically binding to a nucleic acid of MTC and/or Mycobacterium tuberculosis is used.
vi. The method according to any one of (i) to (v), wherein at least one probe specifically binding to a nucleic acid of MTC and/or Mycobacterium tuberculosis binds an amplification product obtained with the primer sets in the preceding claims, said primers being capable of amplifying fragments of the lepA gene and the gyrB gene, respectively. The gyrB gene and lepA gene, respectively, have been deposited in GenBank (GenBank ID: BX842572.1; GI: 41352722 and GenBank ID: CP001642.1; GI: 339296727, respectively) and are shown in SEQ ID Nos: 10 and 11, respectively.
vii. The method according to any one of (i) to (vi), wherein the at least one probe is selected from the oligonucleotides set forth in SEQ ID Nos: 3 and/or 6.
viii. The method according to any one of (i) to (vii), wherein detection of the amplification product obtainable with the oligonucleotide primers set forth in SEQ ID Nos: 4 and 5 or with the oligonucleotide primers set forth in SEQ ID Nos.: 14 and 15 using the probe according to SEQ ID NO: 6 is indicative of M. tuberculosis.
ix. The method according to any one of (i) to (viii), wherein the primers and or probes carry a fluorescent moiety.
x. A method for the diagnosis of a MTC and/or Mycobacterium tuberculosis infection comprising performing one of the methods according to any one of the preceding embodiments.
xi. A method for monitoring the treatment of MTC and/or Mycobacterium tuberculosis infection, said method comprising performing the method according to embodiment (x) before treatment with at least one antibiotic drug and during and/or after treatment with said drug.
xii. An assay for detection of at least nucleic acid of MTC and/or Mycobacterium tuberculosis in a biological sample comprising primers specifically hybridizing to said nucleic acid derived from said Mycobacterium tuberculosis, wherein said assay is suitable for real-time PCR.
xiii. The assay according to (xii), wherein the assay comprises primers and/or probes set forth in any one of the preceding embodiments.
xiv. The assay according to embodiment (xii) or (xiii), wherein the assay is adapted for use in a fully automated laboratory.
xv. A diagnostic composition comprising primers and/or probes set forth in any one of any one of the preceding embodiments.
xvi. A diagnostic kit for the detection of Mycobacterium tuberculosis in a biological sample comprising primers and/or probes set forth in any one of the preceding embodiments, and optionally comprising instructions for use.
xvii. The diagnostic kit according to embodiment (xvi), wherein said kit further comprises enzymes, deoxynucleotides, and/or buffers for performing a reverse transcription step and/or a PCR step.
xviii. The diagnostic kit according to any one of embodiments (xvi) or (xvii) further comprising reagents for the isolation of nucleic acids from a biological sample.
xix. Further, the methods, kits or assays may also comprise steps or reactions serving as positive controls, negative controls or extraction controls, to monitor the specificity or sensitivity of the methods, kits or assays. Primers and probes serving for the amplification and detection of extraction controls are disclosed, e.g. in SEQ ID Nos: 16, 17 and 18 or in SEQ ID Nos: 7, 8 and 9.

### SUMMARY

The invention provides for methods of identifying MTC and/or Mycobacterium tuberculosis by real-time polymerase chain reaction (PCR) in a biological sample.

Primers and probes for detecting MTC and/or Mycobacterium tuberculosis are also provided by the invention, as are kits or compositions containing such primers and probes.

Methods of the invention can be used to identify nucleic acids from specimens for diagnosis of MTC and/or Mycobacterium tuberculosis infection. The specific primers and probes of the invention that are used in these methods allow for the amplification and monitoring the development of specific amplification products.

In particular an assay for MTC and/or Mycobacterium tuberculosis is provided, which allows for detection and/or diagnosis of MTC and/or Mycobacterium tuberculosis, and for distinguishing tuberculosis from other mycobacterial species belonging to the MTC.

According to one aspect of the invention, a method for detecting the presence or absence of MTC and/or Mycobacterium tuberculosis in a biological sample from an individual is provided. The method may comprise a reverse transcription step, at least one cycling step, which includes an amplifying step and a hybridizing step. The amplifying step includes contacting the sample with at least one pair of specific primers to produce an amplification product, if an MTC and/or Mycobacterium tuberculosis-derived nucleic acid molecule is present in the sample. The hybridization step includes contacting the sample with MTC and/or Mycobacterium tuberculosis-specific probes. In the assays of the present invention primer pairs are used that are suitable to specifically hybridize to nucleic acids of MTC/Mtb, respectively, but not to other nucleic acids of other pathogens causing respiratory infections. As a result of the methods described herein, the simultaneous amplification and subsequent detection of the target pathogen, and in particular the distinction of M. tuberculosis from other bacteria belonging to MTC is possible. A pair of primers comprises a first primer and a second primer. Sequences of primers and probes of the invention are shown in the sequence listing.

In some aspects of the invention, the primers and/or probes of the invention can be labeled with a fluorescent moiety. Fluorescent moieties for use in real-time PCR detection are known to persons skilled in the art and are available from various commercial sources, e.g. from Life Technologies™ or other suppliers of ingredients for real-time PCR.

Representative biological samples from the respiratory tract include throat swabs, throat washings, nasal swabs, and specimens from the lower respiratory tract. In addition, the cycling step can be performed on a control sample. A control sample can include the same portion of the target MTR and/or Mycobacterium tuberculosis-derived nucleic acid molecule. Alternatively, a control sample can include a nucleic acid molecule other than an MTR and/or Mycobacterium tuberculosis-nucleic acid molecule.

Cycling steps can be performed on such a control sample using a pair of control primers and a pair of control probes. The control primers and probes can be different from Mycobacterium tuberculosis primers and probes.

One or more amplifying steps produce a control amplification product. Each of the control probes hybridizes to the control amplification product.

In another aspect of the invention, there are provided articles of manufacture, or kits.

Kits of the invention can include at least one pair of specific primers for the amplification of MTR and/or Mycobacterium tuberculosis- and at least one MTR and/or Mycobacterium tuberculosis-probe hybridizing specifically with the amplification products. Preferably, the Mtb-specific probe distinguishes this bacterium from other bacteria of the MTC:

Articles of manufacture can include fluorophoric moieties for labeling the primers or probes or the primers and probes are already labeled with donor and corresponding acceptor fluorescent moieties.

The article of manufacture can also include a package insert having instructions thereon for using the primers, probes, and fluorophoric moieties to detect the presence or absence of MTR and/or Mycobacterium tuberculosis in a sample.

In another aspect of the invention, there is provided a method for detecting the presence or absence of MTR and/or Mycobacterium tuberculosis in a biological sample from an individual. Such a method includes performing at least one cycling step. A cycling step includes at least one amplifying step and a hybridizing step. Generally, an amplifying step includes contacting the sample with a pair of primers to produce an amplification product if a MTR and/or Mycobacterium tuberculosis-derived nucleic acid molecule is present in the sample. Generally, a hybridizing step includes contacting the sample with a MTR and/or Mycobacterium tuberculosis-specific probe. The probe is usually labeled with at least one fluorescent moiety. The presence or absence of fluorescence is indicative of the presence or absence of MTR and/or Mycobacterium tuberculosis in said sample.

Amplification generally involves the use of a polymerase enzyme. Suitable enzymes are known in the art, e.g. Taq Polymerase, etc.

In another aspect of the invention, there is provided a method for detecting the presence or absence of MTR and/or Mycobacterium tuberculosis in a biological sample from an individual. Such a method includes performing at least one cycling step. A cycling step can include an amplifying step and a dye-binding step. An amplifying step generally includes contacting the sample with a pair of MTR and/or Mycobacterium tuberculosis-specific primers to produce a MTR and/or Mycobacterium tuberculosis-specific amplification product if a MTR and/or Mycobacterium tuberculosis nucleic acid molecule is present in the sample. A dye-binding step generally includes contacting the amplification products with a double-stranded DNA binding dye. The method further includes detecting the presence or absence of binding of the double-stranded DNA binding dye into the amplification product. According to the invention, the presence of binding is typically indicative of the presence of MTR and/or Mycobacterium tuberculosis nucleic acid in the sample, and the absence of binding is typically indicative of the absence of MTR and/or Mycobacterium tuberculosis nucleic acid in the sample. Such a method can further include the steps of determining the melting temperature between the amplification product and the double-stranded DNA binding dye. Generally, the melting temperature confirms the presence or absence of MTR and/or Mycobacterium tuberculosis nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for Mycobacterium tuberculosis.

Treatment for Mycobacterium tuberculosis can include, e.g., administration of an antibiotic (e.g. isoniazid or rifampicin) to the individual.

The invention also provides for the use of the articles of manufacture described herein to determine whether or not an individual is in need of treatment for Mycobacterium tuberculosis.

Further, the methods and/or the articles of manufacture described herein can be used to monitor an individual for the effectiveness of a treatment for Mycobacterium tuberculosis as well as in epidemiology to monitor the transmission and progression of Mycobacterium tuberculosis from individuals to individuals in a population. The methods and/or the articles of manufacture (e.g., kits) disclosed herein can be used to determine whether or not a patient is in need of treatment for Mycobacterium tuberculosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting MTR and/or Mycobacterium tuberculosis nucleic in a biological sample that is more sensitive and specific than existing assays is described herein.

Primers and probes for detecting MTR and/or Mycobacterium tuberculosis infections and articles of manufacture containing such primers and probes are also provided. The increased sensitivity of real-time PCR for detection of MTR and/or Mycobacterium tuberculosis as well as the improved features of real-time PCR including sample containment and real-time detection of the amplified product, make feasible the implementation of this technology for routine diagnosis of MTR and/or Mycobacterium tuberculosis infections and the distinction of Mtb-infections from infections with other bacteria belonging to the MTC in the clinical laboratory.

The invention provides methods to detect MTR and/or Mycobacterium tuberculosis by amplifying, for example, a portion of an nucleic acids derived from MTR and/or Mycobacterium tuberculosis. Nucleic acid sequences from Mycobacterium tuberculosis are available, e.g. in GenBank. Mycobacterium tuberculosis can be ordered from commercial sources, e.g. from the ATCC (http://www.atcc.org/).

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis.

Oligonucleotide probes are generally 15 to 30 nucleotides in length. Primers useful within the context of the present invention include oligonucleotides suitable in PCR reactions for the amplification of nucleic acids derived from Mycobacterium tuberculosis.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise.

A “homologous” oligonucleotide is a primer or probe that has at least 80%, 90%, 95%, 96%, 97%, 98% or 99% nucleotide sequence identity and maintains the function of a primer as a replication initiation molecule or of a probe as a detection molecule specifically binding to, e.g. the amplification product using the primer disclosed herein. Such homologous oligonucleotides may also contain one or more nucleotides that are different from dideoxynucleotides, e.g. artificial nucleotides or ribonucleotides.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

A “complementary” oligonucleotide, e.g. of a primer or probe corresponds to the antisense counterpart of a given oligonucleotide. That is, “A” is complementary to “T” and “G” to “C” and vice versa. Of course, this applies also to non-natural analogs of deoxynucleotides, as long as they are capable of “base-pairing” with their counterparts.

As used herein, the term “target amplification” refers to enzyme-mediated procedures that are capable of producing billions of copies of nucleic acid target. Examples of enzyme-mediated target amplification procedures known in the art include PCR.

Within the context of the present invention, the nucleic acid “target” is the nucleic acid sequence of MTR and/or Mycobacterium tuberculosis.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates.

Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80%, 90%, 95%, 96%, 97%, 98%, or 99% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of MTR and/or Mycobacterium tuberculosis-specific nucleic acids. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The amplification primers and detection probes of the present invention are set forth in the sequence listing.

In one aspect of the invention, there is provided a method for detection of MTR and/or Mycobacterium tuberculosis in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detection of MTR and/or Mycobacterium tuberculosis in a sample comprising the steps of obtaining a tissue sample from a patient; extracting nucleic acids from the sample; amplifying the nucleic acid, wherein the DNA/RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, sputum, and nasopharyngeal washes.

In another embodiment of the invention, the assay is a component of a devices that is suitable in fully automated laboratories capable of extracting nucleic acids from a sample (e.g. using the epMotion System of Eppendorf International or the Sentosa platform of Vela Diagnostics), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXAMPLES

### Example 1

### Detection of MTC and M. tuberculosis gDNA

Genomic DNA was isolated according to standard protocols and real-time PCR was performed with primers specific for the lepA gene (primers are depicted in SEQ ID Nos: 1 and 2) and with primers with the M. tuberculosis specific gyrB gene (these primers are depicted in SEQ ID Nos: 4 and 5). Amplification products were detected with specific TaqMan® Probes for lepA and gyrB depicted in SEQ ID Nos: 3 and 6, respectively. As a control for successful extraction of genomic DNA, control DNA derived from Lactococcis lactis was added to the samples and a fragment of the HtrA gene of said organism was co-amplified and detected with a specific probe (the HtrA primers and probe are depicted in SEQ ID Nos.: 7 to 9). Preferably, the extraction control primers and probes are those disclosed in SEQ ID Nos: 16 to 18.

Table 1 below shows that the detection of amplification products of lepA, gyrB and HtrA (Extraction control, EC) was possible when only 10 copies of DNA were present in the reaction tubes, as indicated by the Cₜ value of ≦40.

TABLE 1

| Copy/rxn | lepA | gyrB | EC |
|---|---|---|---|
| 1 | — | — | 30.75 |
| 2 | — | — | 30.71 |
| 5 | — | 36.29 | 30.91 |
| 10 | 37.59 | 37.52 | 30.57 |
| 10² | 34.82 | 34.05 | 30.64 |
| 10³ | 30.91 | 31.04 | 31.22 |
| 10⁴ | 27.13 | 27.79 | 32.70 |
| 10⁵ | 23.13 | 24.26 | 41.94 |
| 10⁶ | 20.11 | 21.39 | — |
| 10⁷ | 16.80 | 17.75 | — |
| 10⁸ | 13.07 | 14.48 | — |

Table 2 shows the high specificity of the primers and probes for bacteria belonging to the MTC (Mycobacterium tuberculosis and M. bovis) as shown by a “+” in the column entitled lepA. The amplification of gyrB, which is specific for M. tuberculosis, was possible with DNA extracted from said organism. DNA extracted from other pathogens never yielded amplification products for any of lepA and gyrB. That this is not due to failure in the extraction of DNA is shown in the column entitled EC (Extraction control), which was always positive.

TABLE 2
Specificity Testing (EC-extraction control)

| Pathogen | lepA | gyrB | EC |
|---|---|---|---|
| Mycobacterium tuberculosis gDNA | + | + | + |
| Mycobacterium bovis gDNA | + | − | + |
| Mycobacterium phlei gDNA | − | − | + |
| Bordetella pertussis gDNA | − | − | + |
| Campylobacter jejuni subsp. Jejuni gDNA | − | − | + |
| Chlamydia trachomatis, UW-57/Cx | − | − | + |
| Clostridium difficile gDNA, strain 630 | − | − | + |
| Clostridium difficile gDNA, strain 90556-M6S | − | − | + |
| Enterococcus faecalis gDNA, strain V583 | − | − | + |
| Enterococcus faecium gDNA, strain MMC4 | − | − | + |
| Giardia intestinalis gDNA, WB clone C6 | − | − | + |
| Klebsiella pneumoniae gDNA, strain ART2008133 | − | − | + |
| Neisseria meningitidis gDNA, strain M1883 | − | − | + |
| Pseudomonas aeruginosa gDNA, strain Boston 41501 | − | − | + |
| Salmonella enterica ssp. Diarizonae, strain MZ1444 | − | − | + |
| Serratia mercescens gDNA, CDC3100-71 | − | − | + |
| Staphylococcus epidermidis gDNA | − | − | + |
| Streptococcus agalactiae gDNA, strain 2603V/R | − | − | + |
| Streptococcus mutans gDNA | − | − | + |
| Streptococcus sanguinis gDNA | − | − | + |
| Moraxella catarrhalis, strain 20 | − | − | + |
| Neisseria gonorrhoeae | − | − | + |
| Neisseria sicca | − | − | + |
| Salmonella enterica ssp. Enterica serovar | − | − | + |
| Typhimurium, ATCC 14028 | | | |
| Streptococcus pneumoniae | − | − | + |
| Streptococcus pyogenes Rosenbach | − | − | + |

The above data demonstrate the high specificity and sensitivity of the assay according to the invention.

### Example 2

A second real-time polymerase chain reaction (PCR)-based in vitro diagnostic test, for the qualitative detection of MTC species (Mycobacterium tuberculosis, M. bovis, M. bovis BCG), and differentiation between M. tuberculosis and other MTC species in sputum samples was developed. This in vitro diagnostic test was tested on the Sentosa SX101 instrument, with the Sentosa SX Bacteria gDNA Kit v2.0, in conjunction with the Rotor-Gene Q MDX 5plex HRM instrument. Test results may be used as supplementary data for diagnosis, or large scale screening of Mycobacteria infection. The test comprises a ready-to-use system for the detection of Mycobacteria DNA using PCR on the Rotor-Gene Q MDX 5plex HRM instrument, with sample preparation and assay set-up using the Sentosa SX101 instrument. The MTC master mix contains reagents and enzymes for the specific amplification of a 88 bp fragment of gDNA common to all MTC species and 167 bp fragment of gDNA specific to M. tuberculosis, and for the direct detection of the specific amplicon in the fluorescence channels Cycling Yellow and Cycling Green of the Rotor-Gene Q MDX 5plex HRM instrument. In addition, the test contains a second set of primers/probes designed to detect an extraction control (EC1) target in the fluorescence channel Cycling Red. This extraction control can be used as a control for the sample preparation procedure and as a PCR inhibition control. This second amplification system does not reduce the detection limit of the analytical MTC PCR. The test also contains a positive control (PC) and a negative control (NC) that allows the user to assess whether the PCR reaction has been performed properly. Each set of 8 samples will include 1 NC, 1 PC and 6 clinical samples. The primers and TaqMan® probes in the table below were used (SEQ ID Nos: 12-18, and 3 and 6):

TABLE 3

| | | 5′ | 3′ |
|---|---|---|---|
| Oligo | Sequence | Modifi- | Modifi- |
| Name | (5′-3′) | cation | cation |
| MTC_lepA_ | CGTAGACCGTGCGGATCTTG | — | — |
| F01_L4 | | | |
| MTC_lepA_ | CCTGCTGCACATGGAGATCAC | — | — |
| R01_L3 | | | |
| MTB_gyrB_ | GCAGAAGGTCTGTAACGAACAG | — | — |
| F01_L3 | | | |
| MTB_gyrB_ | CGAGGACACAGCCTTGTTCAC | — | — |
| R01_L3 | | | |
| MTC_lepA_ | CGGATTTCGGTGCCGTCGTCTTTG | JOE | BHQ-1 |
| P02 | | | |
| MTB_gyrB_ | CACCGACGCGAAAGTCG | 6-FAM | BHQ-1 |
| P02_S2 | | | |
| EC_RNA_ | AATTCCTCAGGGATTATTGTGGC | — | — |
| F12 | | | |
| EC_RNA_ | CACAACCAGTATGCAACATCAACC | — | — |
| R12 | | | |
| EC_RNA_ | AACCTCTTGAACTGACAGCGTGTGC | Quasar | BHQ- |
| P11 | | 670 | 2 |

### Performance Characteristics

### Analytical Sensitivity

The analytical limit of detection (LoD) was assessed for the PCR test using M. tuberculosis genomic DNA (strain H37Rv) to determine LoD for the Sentosa SA MTC PCR Test and M. bovis BCG (strain TMC 1011 Pasteur) to determine LoD for the whole workflow of Sentosa System and Sentosa SA MTC PCR Test. Serial dilutions of gDNA and bacterial culture were performed. The LoD was the dilution giving a final sample detection ≧95% (Table 1).

TABLE 4
Limit of detection fortest.

| | PCR test only | | Whole workflow | |
|---|---|---|---|---|
| | (copies/μL) | | (copies/μL) | |
| MTC strain | LoD | 95% CI | LoD | 95% CI |
| M. tuberculosis, | 1.0 | 0.51 − 1.05 | N/A | N/A |
| H37Rv | | | | |
| M. bovis BCG, TMC | N/A | N/A | 2.6 | 0.61 − 3.59 |
| 1011 Pasteur | | | | |

### Analytical Reactivity and Specificity

Analytical reactivity of the PCR Test was ensured by the selection of primers, probes and stringent reaction conditions. The primers and probes were checked for possible homologies to all sequences published in public databases by sequence comparison analysis. The detectability of all relevant genotypes has thus been ensured by database alignment and PCR run on Rotor-Gene Q MDX 5plex HRM instrument with successful detection of the MTC strains, as shown in Table 5 (Detection of MTC strains “+” shows signal detection and “−” shows no signal detection).

TABLE 5

| | | | | Extrac- |
|---|---|---|---|---|
| | | | | tion |
| | | M.tb | MTC | control |
| | | detection | detection | detection |
| | | (Green | (Yellow | (Red |
| Mycobacteria, strain | Source | channel) | channel) | channel) |
| Mycobacterium | ATCC | + | + | + |
| tuberculosis, H37Rv | | | | |
| Mycobacterium | ATCC | + | + | + |
| tuberculosis, H37Ra | | | | |
| Mycobacterium bovis BCG, | ATCC | − | + | + |
| TMC 1011 Pasteur | | | | |

Analytical specificity of the test was assessed using the control group listed in Table 6. None of the tested pathogens were reactive, confirming the specificity of the Test for MTC.

TABLE 6

| | | | | Extrac- |
|---|---|---|---|---|
| | | M.tb | MTC | tion |
| | | detection | detection | control |
| | | (Green | (Yellow | (Red |
| Organism | Source | channel) | channel) | channel) |
| Mycobacterium abscessus | DSMZ | − | − | + |
| Mycobacterium avium | ATCC | − | − | + |
| Mycobacterium gastri | DSMZ | − | − | + |
| Mycobacterium kansasii | ATCC | − | − | + |
| Mycobacterium smegmatis | ATCC | − | − | + |
| Bacillus cereus | ATCC | − | − | + |
| Bordetella pertussis | ATCC | − | − | + |
| Burkholderia cepacia | ATCC | − | − | + |
| Chlamydophila pneumoniae | ATCC | − | − | + |
| Corynebacterium diphtheriae | ATCC | − | − | + |
| Clostridium difficile | ATCC | − | − | + |
| Enterobacter aerogenes | ATCC | − | − | + |
| Escherichia coli | ATCC | − | − | + |
| Haemophilus influenza | ATCC | − | − | + |
| Haemophilus parainfluenzae | ATCC | − | − | + |
| Klebsiella pneumoniae | ATCC | − | − | + |
| Legionella pneumophila | ATCC | − | − | + |
| Moraxella catarrhalis | ATCC | − | − | + |
| Neisseria sicca | ATCC | − | − | + |
| Neisseria meningitidis | ATCC | − | − | + |
| Pseudomonas aeruginosa | ATCC | − | − | + |
| Staphylococcus aureus | ATCC | − | − | + |
| Staphylococcus epidermidis | ATCC | − | − | + |
| Streptococcus pneumoniae | ATCC | − | − | + |
| Streptococcus pyogenes | ATCC | − | − | + |
| Streptococcus anginosus | ATCC | − | − | + |
| Streptococcus salivarius | ATCC | − | − | + |
| Aspergillus niger | ATCC | − | − | + |
| Cryptococcus neoformans | ATCC | − | − | + |
| Adenovirus | ATCC | − | − | + |
| Human Influenzae Virus | ATCC | − | − | + |
| Type A | | | | |
| Human Influenzae Virus | Vircell | − | − | + |
| Type B | | | | |
| Human Parainfluenzae Type 1 | ATCC | − | − | + |
| Human Parainfluenzae Type 2 | ATCC | − | − | + |
| Human Parainfluenzae Type 3 | ATCC | − | − | + |
| Respiratory Syncytial Virus | ATCC | − | − | + |
| Rhinovirus 87 | ATCC | − | − | + |

Absence of cross-reactivity for other pathogens tested. “+” shows signal detection and “−” shows no signal detection.

### Reproducibility

Reproducibility data permit a regular performance assessment of the test, as well as an efficiency comparison with other products. The inter/intra-assay reproducibility was determined by testing two concentrations of M. tuberculosis gDNA and negative samples in multiple replicates for 5 days by 2 operators using three sets of Sentosa System and two lots of PCR tests. The overall reproducibility assessment setup allows to test intra-assay variability (variability of multiple results for samples of the same concentration within one experiment), the inter-assay variability (variability of multiple results of the assay generated on different instruments of the same type by different operators within one laboratory) and the inter-batch variability (variability of multiple results of the assay using various batches of master mix and primer/probe mix). Overall, tests consistently gave 100% reproducibility with the herein described PCR Test (1440 data points have been analyzed).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the detection of the presence or absence of at least one nucleic acid of bacteria belonging to the Mycobacterium tuberculosis complex (MTC) and/or Mycobacterium tuberculosis in a biological sample, wherein the method comprises:
(a) isolating nucleic acids from the biological sample and optionally performing a reverse transcription step, and
(b) conducting real-time PCR wherein primer sets specific for MTC and/or Mycobacterium tuberculosis nucleic acids are used, said specific primer sets comprising oligonucleotide sequences set forth in SEQ ID NOs: 1 and 2 or in SEQ ID NOs: 12 and 13, and SEQ ID NOs: 4 to 6 or in SEQ ID NOs: 14 and 15, or complements thereof, or sequences that are at least 90% or 95% identical or homologous to SEQ ID NOs 1, 2, 4, 5, 6, 12 to 15 or complements, and further wherein at least one probe specifically binding to a nucleic acid of MTC and/or Mycobacterium tuberculosis is used, said at least one probe being selected from the oligonucleotides set forth in SEQ ID NOs: 3 and/or 6, or sequences that are at least 90% or 95% identical or homologous to SEQ ID Nos NOs: 3 or 6 or complements.

2. The method according to claim 1, wherein the primers and or probes carry a fluorescent moiety.

3. An in vitro method for the diagnosis of a MTC and/or Mycobacterium tuberculosis infection in a subject comprising performing the method according to claim 1.

4. A method for monitoring the treatment of MTC and/or Mycobacterium tuberculosis infection, said method comprising performing the method according to claim 3 before treatment with at least one antibiotic drug and during and/or after treatment with said drug.

5. A real-time PCR assay for the detection of at least one nucleic acid of MTC and/or Mycobacterium tuberculosis in a biological sample comprising primers and/or probes having oligonucleotide sequences as set forth in claim 1.

6. The assay according to claim 5, wherein the assay is adapted for use in a fully automated laboratory.

7. A composition comprising primers and/or probes having oligonucleotide sequences as set forth in claim 1.

8. A diagnostic kit for the detection of Mycobacterium tuberculosis in a biological sample comprising primers and/or probes having oligonucleotide sequences as set forth in claim 1, further comprising instructions for use.

9. The diagnostic kit according to claim 8, wherein said kit further comprises enzymes, deoxynucleotides, and/or buffers for performing a reverse transcription step and/or a PCR step.

10. The diagnostic kit according to claim 8, further comprising reagents for the isolation of nucleic acids from a biological sample.
